(19) United States
(12) Patent Application Publication
(10) Pub. No.: US 2022/0008561 A1
(43) Pub. Date: Jan. 13, 2022

(54) BISMUTH METAL-ORGANIC FRAMEWORKS FOR USE AS X-RAY COMPUTED TOMOGRAPHY CONTRAST AGENTS

(71) Applicants: Northwestern University, Evanston, IL (US); NANJING TECH UNIVERSITY, NANJING SHI (CN)

(72) Inventors: Omar K. Farha, Glenview, IL (US); Lin Zhang, Nanjing (CN); Lee N. Robison, Evanston, IL (US)

(21) Appl. No.: 17/293,176

(22) PCT Filed: Nov. 13, 2019
(86) PCT No.: PCT/US19/61069

§ 371 Date: May 12, 2021

Related U.S. Application Data
(60) Provisional application No. 62/760,201, filed on Nov. 13, 2018.

(51) Int. Cl. A61K 49/04; A61B 6/00

(52) U.S. Cl. CPC A61K 49/04; A61B 6/481

(57) ABSTRACT

Metal-organic frameworks with bismuth cluster nodes (Bi-MOFs) and methods of using the Bi-MOFs as contrast agents in medical imaging systems, such as computerized tomography (CT) systems, are provided. Contrast compositions that include the Bi-MOFs in a carrier in forms suitable for administration to a patient are also provided. The Bi-MOFs include those with Bi₆ nodes connected by multitopic organic linkers.

### CROSS-REFERENCE TO RELATED APPLICATIONS

The present application claims priority to U.S. provisional patent application No. 62/760,201 that was filed Nov. 13, 2018, the entire contents of which are incorporated herein by reference.

### BACKGROUND

X-ray computed tomography (CT) is a non-invasive medical imaging technique that allows for three-dimensional (3D) visualization of internal organs and tissues such as the liver, lungs, bone, cardiovascular system, and gastrointestinal system.

Contrast media are typically used for medical diagnostic imaging, including CT imaging, to increase the intensity difference between the tissue of interest and other tissues. To be feasible for clinical use, a CT contrast agent should require the lowest dose possible, produce the maximum contrast between the tissue of interest and background scattering events, and be minimally toxic to patients. Commercially available CT contrast agents are based on small molecules composed of either iodine or barium. Unfortunately, the most widely used CT contrast agents often display only two of these three desirable characteristics. High doses of iodine have been known to induce immediate allergic reaction and/or cardiac, endocrine, and renal complications. Similarly, typically administered doses of a barium-based contrast agents can produce side effects including allergic reactions and mild to severe stomach cramping and/or diarrhea.

The performance of a CT contrast agent can be predicted by considering the mass absorption coefficient, μ, determined using eqn. 1:

μ≈(pZ⁴)/(EA³) Equation 1:

where ρ is the material density, Z is the atomic number, A is the atomic mass, and E is the energy of X-rays. (See, e.g., H. Lusic, et al. Chem. Rev., 2013, 113, 10.1021/cr200358s.) The Z⁴ term yields a significant contrast difference between the CT agent and the surrounding tissue, as contrast enhancement is largely due to the photoelectron effect. Given this fact, one can infer the use of iodine and barium CT-agents is based on their overall safety and cost rather than on their efficiency to attenuate X-rays.

Bismuth nanoparticles, bismuth-carbon nanotubes, and bismuth coordination polymers have been proposed for use in CT imaging applications. (See, e.g., O. Rabin, et al., Nat. Mater., 2006, 5, 118; P. C. Naha, et al., J. Mater. Chem. B, 2014, 2, 8239-8248; M. Hernández-Rivera, I. Kumar, et al., ACS Appl. Mat. Interfaces, 2017, 9, 5709-5716; and V. S. Perera, et al., Inorg. Chem., 2011, 50, 7910-7912.)

In addition, several different categories of nanomaterials for next-generation CT-contrast agents have been investigated, including metal-organic frameworks (MOFs). (See, deKrafft et al., J. Mater. Chem. 201222(35): 18139-18144.) MOFs are a class of porous nanomaterials having inorganic nodes and multitopic organic linkers that assemble through coordination bonds into multidimensional periodic lattices. (See, e.g., H. Li, et al., Nature, 1999, 402, 276-279.)

### BRIEF DESCRIPTION OF THE DRAWINGS

Illustrative embodiments of the invention will hereafter be described with reference to the accompanying drawings, wherein like numerals denote like elements.

FIGS. 1A-1B show (FIG. 1A) a Bi₆ node (FIG. 1B) a 1,3,6,8-tetrakis(p-benzoate)pyrene linker used to construct Bi-NU-901. FIG. 1C shows the structure of Bi-NU-901.

FIG. 2A shows an experimental powder X-ray diffraction (PXRD) pattern of Bi-NU-901, in agreement with the simulated pattern of Bi-NU-901 PXRD. FIG. 2B shows N₂ isotherms of Bi-NU-901 based on the volume. FIG. 2C shows pore size distribution of Bi-NU-901 calculated by the density functional theory (DFT) model.

FIG. 3 shows a view down the b-axis of the simulated Bi-NU-901 MOF. The (001) distance is shown by the black arrow.

FIG. 4A shows X-ray attenuation as a function of [Bi/Zr/I/Ba] for Bi-NU-901, Zr-NU-901, Iodixanol, and barium sulfate at 35 kVp. FIG. 4B shows X-ray attenuation as a function of [Bi/Zr/I/Ba] for Bi-NU-901, Zr-NU-901, Iodixanol, and barium sulfate at 50 kVp.

### DETAILED DESCRIPTION

Metal-organic frameworks with bismuth nodes (Bi-MOFs) and methods of using the Bi-MOFs as contrast agents in CT imaging systems are provided.

MOFs are hybrid, crystalline, porous compounds made from metal-ligand networks that include inorganic nodes connected by coordination bonds to organic linkers. The inorganic nodes or vertices in the framework are composed of metal ions or clusters. For example, the inorganic nodes may have 6 metal atoms. Such nodes are generally designated M₆ nodes; for example, a node with 6 bismuth atoms would be designated a Bi₆ node. In a Bi-MOF, the nodes comprise bismuth ions or clusters of ions.

The Bi-MOFs are able to provide good contrast intensities in CT imaging and diagnostic applications, can be used at low doses relative to conventional CT contrast agents, and are non-toxic. The use of bismuth-based MOFs is advantageous because they are synthetically accessible, and bismuth is a non-radioactive element with a high atomic number, affording it better X-ray attenuation properties than iodine and barium-based CT contrast agents. Additionally, bismuth is non-toxic to humans. The Bi-MOFs can be synthesized with nanoscale dimensions, so that the Bi-MOFs do not diffuse to extravascular spaces or undergo rapid renal clearance, which is advantageous for intravenous delivery.

As used herein, the phrases bismuth-based MOF and Bi-MOF refer to MOFs that permanently porous structures, characterized in that they show N₂ isotherms and retain their porous structure even when the organic solvent it removed (e.g., when they are dried after synthesis). Useful Bi-MOFs include microporous Bi-MOFs with type-I N₂ isotherms.

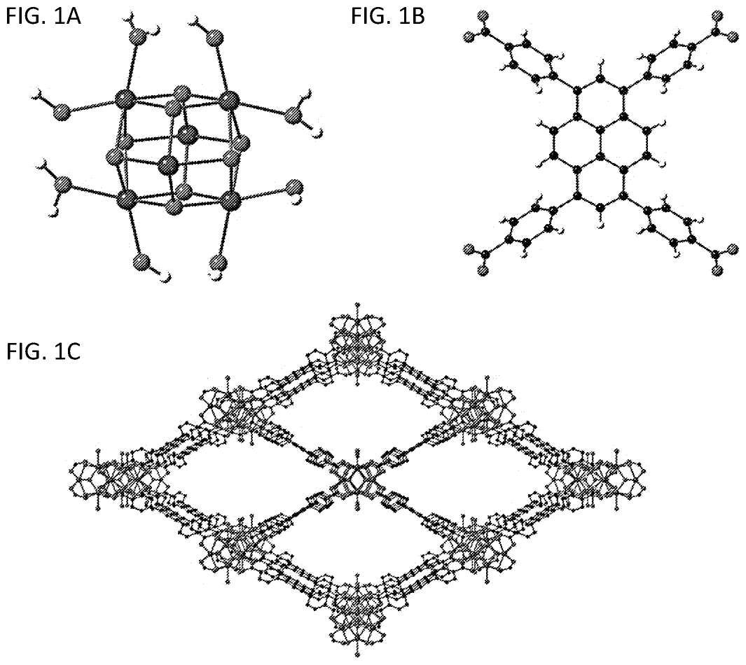

The Bi-MOFs include cluster-based Bi-MOFs having Bi₆ nodes (FIG. 1A) connected by multitopic linkers, such as tetratopic linkers. Some such MOFs include tetratopic linkers containing pyrene groups (FIG. 1B) or biphenyl groups. The structure of one such Bi-MOF is shown in FIG. 1C. This Bi-MOF has Bi₆ nodes connected by tetratopic 1,3,6,8-tetrakis(p-benzoate)pyrene linkers and has an 8-connected scu network topology. More details regarding the fabrication of this MOF are provided in the Example. Another Bi-MOF having Bi₆ nodes connected by tetratopic 4,4′,4″,4′″-(pyrene-1,3,6,8-tetrayl)tetrabenzoic acid (TBAPy) linkers has a csq network topology and is isostructural with the Zr₆ MOF, NU-1000 described in Mondloch, et al., J. Am. Chem. Soc. 135, 10294_10297 (2013). Bismuth based MOFs having Bi₆ nodes connected by tetratopic 3,3′,5,5′-tetrakis(4-carboxyphenyl)-1,1′-biphenyl (TCPB) linkers with a csq network topology can also be used. The above-mentioned MOFs can be made using the same synthesis methods (for example, solvothermal syntheses) that are used to make their isostructural counterparts (for example, MOFs having the same linkers and network topologies, but different metal nodes), by replacing the metal salts using in those syntheses with corresponding bismuth salts. The Bi-MOFs can also have tritopic or tetratopic carboxylic acid linkers, such as those described in Cryst. Growth Des. 2018, 18, 7, 4060-4067, and other multitopic linkers, including those described in Chem. Soc. Rev., 2012, 41, 1088-1110.

Still other Bi-MOFs that can be used as the contrast agents include those having triazine tribenzoic acid linkers (e.g., triazine-2, 4, 6-triyl-tribenzoic acid linkers), carboxyphenyl benzene linkers (e.g., 1, 2, 4, 5-tetrakis-(4-carboxyphenyl) benzene linkers), or tetracarboxylate linkers (e.g., biphenyl-3,3′,5,5′-tetracarboxylate linkers), such as CAU-7, NOTT-220, CAU-17, CAU-7-TATB, and CAU-35. Descriptions of these can be found in M. Köppen et al., Dalton Trans., 2017, 46, 8658-8663; M. Köppen et al., Cryst. Growth Des., 2018, 18, 4060-4067; and M. Savage et al., Chem. Eur. J., 2014, 20, 8024-8029, the disclosures of which are incorporated herein by reference for descriptions of the structures and methods of synthesizing these MOFs. The list of bismuth MOFs provided here is intended as illustrative and not comprehensive; other Bi-MOFs can be used in the methods described herein.

The Bi-MOFs can be used as contrast agents in X-ray based CT imaging to improve the contrast between biological tissue in which the Bi-MOFs have been taken up and surrounding tissue, thereby increasing CT sensitivity and enhancing the differentiation between the different tissues.

The CT imaging process includes the steps of directing X-rays at biological tissue in which the contrast agent has been taken up from one or more orientations and measuring an attenuation of the X-ray intensity resulting from the passage of the X-rays through the biological tissue along one or more beam paths. Known algorithms can then be used to generate an image of the tissue based on the distribution of X-ray attenuation in the volume of biological tissue being imaged.

The components of one embodiment of an X-ray CT system include one or more X-ray sources configured to direct beams of X-ray radiation to a biological tissue, one or more X-ray detectors configured to (i.e., designed to) detect at least a portion of the X-ray radiation passing through the biological tissue along one or more beam paths in order to measure an attenuation in the X-ray radiation intensity, and a sample support configured to position the biological tissue in the one or more beam paths. The X-ray sources can be of the type normally used in medical imaging, such as X-ray tubes, radioactive isotopes, plasma sources, and synchrotrons. The X-ray detectors also can be of the type normally used in medical imaging, such as synchrotrons, photodiodes, CCD detectors, and flat panel sensors. The X-ray CT system may further include a processor in communication with the one or more X-ray sources and the one or more X-ray detectors. The processor may be configured to process data received from the one or more X-ray detectors, where the data includes X-ray radiation intensity attenuation data. The processor is further configured to generate an image of at least a portion of the biological tissue based on the X-ray radiation intensity attenuation data.

Typically, the biological tissue to be imaged will comprise the biological tissue of a patient, where a patient may be an animal, more specifically a mammal, such as a human, and the imaging will be in vivo. However, in vitro imaging of biological tissue can also be carried out. The biological tissue can be imaged by administering an effective amount of a Bi-MOF to a patient, whereby the Bi-MOF is taken up by at least some of the patient's biological tissues. The contrast agent can be administered, for example, intravenously, orally, or rectally. Dosage forms of the contrast agents include liquid or solid dosages, such as tablets, containing the Bi-MOF, with or without suitable carriers. As used herein, the term carrier refers to a diluent, adjuvant, excipient, or vehicle with which the MOFs are administered to a subject. The carriers are compounds that are non-toxic to the patient and do not have a substantial negative effect on or destroy the contrast-enhancing function of the Bi-MOFs. A carrier may be a liquid, such as saline, citrate buffer, phosphate-buffered saline, HEPES ((4-(2-hydroxyethyl)-1-piperazineethanesulfonic acid) buffer or Tris buffer are preferred carrier(s). However, solid carriers may also be used. By way of illustration, carriers include carbohydrates such as sugars, polysaccharides, and starches. Specific examples include lactose, dextrose, saccharose, cellulose, dextran, carboxydextran, aminated dextran, starch, chitosan, and combinations thereof. The carriers may act as inert fillers or may provide a function; thus, carriers include wetting agents, emulsifying agents, pH buffering agents, antibacterial agents, and antioxidants. Contrast agent compositions that include the MOFs mixed with one or more carriers can be made by combining (e.g., mixing or suspending) the MOF with the carriers.

An effective amount of the Bi-MOF refers to an amount that allows for uptake by a biological tissue in a sufficient quantity to provide the desired imaging contrast. The effective amount for a given tissue sample will depend, at least in part, on the volume of the tissue to be imaged. However, by way of illustration only, effective amounts of the Bi-MOFs can include doses in the range from about 500 mL to 1200 mL of a solution containing about 1 mg of the contrast agent per mL.

Once the contrast agent has been administered, the patient is exposed to incident X-ray radiation, the intensity of which becomes attenuated as it passes through biological tissue. The attenuation of the X-ray radiation is then measured, and an image of the biological tissue corresponding to the attenuation of the X-ray radiation is generated.

The contrast agents and CT systems described herein can be used to image the cells, tissues, and organs of a patient. For example, the organs, vasculature, and/or gastrointestinal tract of a patient can be imaged.

### Example

A new bismuth-based cluster MOF, Bi-NU-901, is reported herein, and its use as an X-ray computed tomography (CT) agent is explored.

### Experimental

Reaction of 1,3,6,8-tetrakis(p-benzoic-acid) (H₄TBPy) with Bi(NO₃)₃.5H₂O in a solution of N, N-dimethylformamide (DMF), ethanol, and trifluoroacetic acid (TFA) at 100° C. for 8 h yields a yellow powder of Bi-NU-901. The bismuth salt solution was prepared by mixing Bi(NO₃)₃.5H₂O (80 mg, 0.16 mmol) and TFA (200 μL, 5.88 mmol) in DMF (10 mL) in a 6-dram vial. The solution was heated at 100° C. for one hour. Upon cooling to room temperature, H₄TBAPy linker (40 mg, 0.06 mmol), as synthesized by an established procedure, and additional DMF (10 mL) were added to the bismuth salt solution. (See, e.g., T. C. Wang, et al., Nat. Prot., 2015, 11, 149.) The resulting pale-yellow solution was sonicated for ten minutes and then added into a 100 mL glass vial with ethanol (40 mL). The vial was placed in an oven at 100° C. for 8 hours, during which time a yellow suspension was formed. The Bi-NU-901 powder was soaked in DMF (25 mL), and the solvent was replaced every two hours over a six-hour period. During each solvent exchange, the material was purified by density separation. (See, e.g., 0. K. Farha, et al., J. Am. Chem. Soc., 2008, 130, 8598-8599.) The crystalline NU-901 phase is denser than a difficult to characterize amorphous phase, allowing for facile separation. The Bi-NU-901 solid residue was then soaked in ethanol (25 mL) twice for 2 hours followed by soaking overnight in ethanol. The ethanol-containing samples were activated by supercritical CO₂ drying (SCD) over a period of eight hours. In this method, the liquid CO₂ was purged under positive pressure for four minutes every two hours. Throughout the entire process, the rate of purging was maintained below the rate of filling. Following the final exchange, the temperature was increased to 40° C. (above the critical temperature for CO₂), and the chamber was slowly vented over a period of 15 hours at a rate of 0.1 cc/min. Bi-NU-901 crystals were then transferred to a pre-weighted sorption analysis tube to collect N₂ isotherm without further activation. Additional details are provided in the Detailed Experimental Section, below.

### Results and Discussion

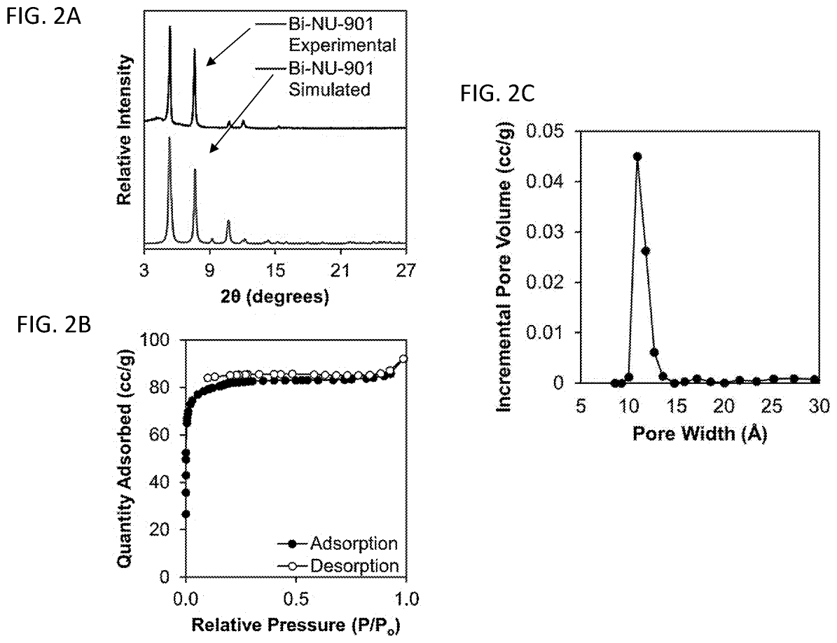

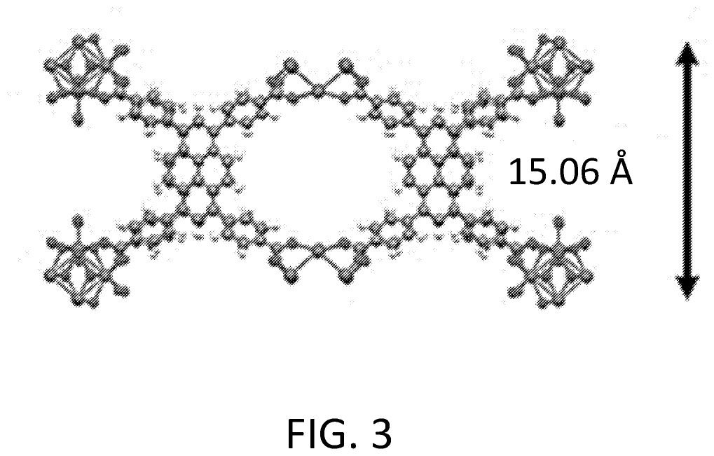

The atomic structure of Bi-NU-901 was simulated based on a combination of the crystal structure of Zr-NU-901 and a modeled [Bi₆O₄(OH)₄(NO₃)₆(H₂O)](H₂O) node. The bulk phase purity of Bi-NU-901 was confirmed by comparing the experimental PXRD pattern with a simulated pattern of Bi-NU-901 and an experimental pattern of Zr-NU-901 (FIG. 2A). The scu topology of the Bi-NU-901 phase features microporous diamond-shaped 1D channels formed by the coordination of Bi₆-nodes to 8 tetratopic H₄TBAPy linkers. Nitrogen adsorption-desorption isotherms collected for activated samples of Bi-NU-901 show a type I isotherm (FIG. 2B), consistent with the microporous structure of the Bi-NU-901, which is also evident from pore size distribution (FIG. 2C). The DFT calculated pore-size distribution revealed one pore with a diameter of ˜11 Å, which corresponds closely to that of Zr-NU-901 (˜12 Å). An average Brunauer-Emmett-Teller (BET) surface area of 320 m²/g was calculated for the material. The determined scu topology was further supported by scanning transmission electron microscopy (STEM) images of Bi-NU-901, from which the d-spacing between metal nodes was calculated (FIG. 3). The experimental distance between the nodes on the (001) plane was measured from the fringe spacing in the image and the associated spots in the Fourier Transform to be ˜15.43 Å, aligning closely with the 15.06 Å d-spacing calculated from the simulated Bi-NU-901 (001) plane. X-ray photoelectron spectroscopy (XPS) confirmed the expected +3 valence of bismuth ions in the MOF node, and the Bi-NU-901 thermal stability was tested using thermogravimetric analysis (TGA). The TGA results showed that Bi-Nu-901 is stable up to 400° C. As revealed by SEM, Bi-NU-901 crystals exhibit average size of ˜7.0 m. Based upon these results and previously reported hexanuclear, 8-connected MOFs, this structure is proposed as Bi₆(μ₃-OH)₈(HCO₂)₂(TBAPy)₂.

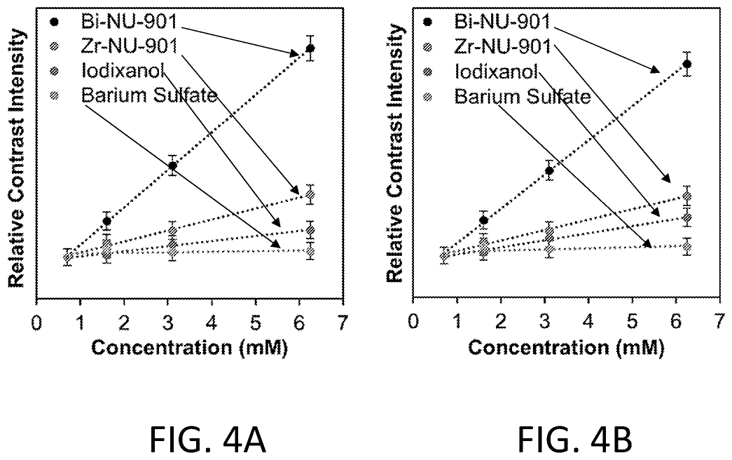

CT measurements were conducted using newly synthesized Bi-NU-901. All imaging samples were prepared by dispersing Bi-NU-901 in a 10% Tween20 surfactant-water solution, and images were obtained at varying concentrations from 0.8-6.25 mM. CT images were obtained at three different X-ray tube voltages: 35 kV, 50 kV, and 70 kV. For comparison, CT images were also collected of Zr-NU-901, the Zr-based analog of Bi-NU-901 with the same topology; Iodixanol, a commercially available iodinated contrast agent; and barium sulfate, the X-ray attenuating element in all barium-based CT-imaging agents. Under all X-ray voltages, Bi-NU-901 outperformed each of the examined CT contrast agents as demonstrated by the plots of X-ray attenuation (Relative Intensity) against the concentration of the respective heavy element. Notably, at 50 kV and a concentration of 6.25 mM, the Bi-NU-901 sample yielded 53% better contrast than Iodixanol, a commonly used commercial CT contrast agent (FIGS. 4A-4B). This energy is closer to the energies used to image the gastrointestinal tract of humans in clinical settings than the lower 35 kVp voltage used. The enhancement in attenuation of the bismuth-based MOF against other CT-contrast agents tested would be even more pronounced at higher X-Ray voltages, such as those used to image human patients (80-120 kVp).

### Detailed Experimental

### Materials

The starting chemical reactants bismuth(III) nitrate pentahydrate (Sigma Aldrich, 99.99%), anhydrous N,N′-dimethylformamide (Aldrich, 99.8%, noted DMF), Reagent alcohol (Sigma Aldrich, <0.0005% water, noted ethanol), trifluoroacetic acid (Sigma Aldrich, ReagentPlus®, 99%, noted TFA), Iodixanol (Sigma Aldrich), barium sulfate (Sigma Aldrich, 99.99%), and TWEEN© 20 (Sigma Aldrich) are commercially available and have been used without any further purification. The ligand 1,3,6,8-tetrakis (p-benzoic acid) pyrene (H₄TBAPy) was synthesized according to a published procedure. (See, e.g., Wang, T. C., et al., Nature protocols 2015, 11, 149.)

### Physical Methods and Measurements

PXRD data were collected at room temperature on a STOE-STADI-P powder diffractometer at Northwestern University's IMSERC facility equipped with an asymmetric curved Germanium monochromator (CuKα1 radiation, λ=1.54056 Å) and a one-dimensional silicon strip detector (MYTHEN2 1K from DECTRIS). The line focused Cu X-ray tube was operated at 40 kV and 40 mA. Powder samples were packed in 3 mm metallic masks and sandwiched between polyimide tape. Intensity data for 20 from 2° to 41° were collected over a period of 7 mins. Prior to measurement, the instrument was calibrated against a NIST Silicon standard (640d).

SEM images were collected on a Hitachi SU8030 FE-SEM (Dallas, Tex.) microscope at Northwestern University's EPIC/NUANCE facility. Before imaging, samples were coated with OsO₄ to ˜10 nm thickness in a Denton Desk III TSC Sputter Coater (Moorestown, N.J.).

The STEM experiments were performed on a JEOL Cs corrected ARM 200 kV (JEOL, Ltd. Akishima, Tokyo, Japan) equipped with a cold field-emission source that generates a nominal 0.1 nm probe size under standard operating conditions. The ARM 200 was operated under low dose conditions to minimize the electron beam damage. All images were acquired in the high angle annular dark field (HAADF) or Z-contrast imaging mode. The samples were prepared by drop casting the mixture of the Bi-NU-901 MOF and ethanol onto the 200-mesh copper TEM grid with lacy carbon film.

N₂ adsorption-desorption isotherms were collected at 77K on a Micromeritics Tristar II 3020 (Micromeritics, Norcross, Ga.). The data points between 0.04 and 0.15 P/P₀ were chosen for BET surface area calculation to minimize the error for consistency criteria (R²=0.9999).

TGA was performed at Northwestern University's Materials Characterization and Imaging facility using a TGA/DCS 1 system (Mettler-Toledo A G, Schwerzenbach, Switzerland) with STARe software. Samples were heated from 25 to 650° C. at a rate of 10° C./min under a constant flow of N₂.

XPS measurements were carried out at the KECK-II/NUANCE facility at Northwestern University on a Thermo Scientific ESCALAB 250 Xi (Al Kα radiation, hv=S5 1486.6 eV) equipped with an electron flood gun. XPS data were analyzed using Thermo Scientific Advantage Data System software and all spectra were referenced to the C1s peak (284.8 eV).

SCD was performed with a Tousimis™ Samdri® PVT-30 critical point dryer. Briefly, the ethanol-containing samples were activated by supercritical C02 drying over a period of eight hours. (See., e.g., Nelson, A. P., et al., Journal of the American Chemical Society 2009, 131, 458-460.) In this method, the liquid C02 was purged under positive pressure for four minutes every two hours. The rate of purging was maintained below the rate of filling. Following the final exchange, the temperature was increased to 40° C. (above the critical temperature for CO₂) and the chamber was vented over a period of 15 hours at a rate of 0.1 cc/min.

CT images were acquired at Northwestern University's Center for Advanced Molecular Imaging (CAMI) with a preclinical micro PET/CT imaging system, Mediso nanoScan scanner (Mediso-USA, Boston, Mass.). Data were acquired with 2.17 magnification, 33 μm focal spot, 1×1 binning, with 720 projection views over a full circle, with a 300 ms exposure time. Three images were acquired, using 35 kVp, 50 kVp, and 70 kVp. The projection data were reconstructed with a voxel size of 68 μm using filtered (Butterworth filter) back-projection software from Mediso. The reconstructed data were analyzed in Amira 6.5 (FEI, Houston, Tex.). Regions of interest were identified for each sample at each energy. The mean image intensity, in Hounsfield Units, was used in the statistical analysis.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A method for imaging a patient, the method comprising:
administering a metal-organic framework comprising bismuth nodes connected by organic linkers to a patient, whereby the metal-organic framework is taken up by biological tissue in the patient;
exposing the patient to incident X-ray radiation;
measuring an attenuation of the X-ray radiation passing through the biological tissue; and
generating an image of a distribution of the X-ray radiation attenuation.

2. The method of claim 1, wherein the bismuth nodes are Bi₆ nodes and the organic linkers comprise pyrene groups.

3. The method of claim 2, wherein the metal-organic framework comprises Bi₆ nodes connected by tetratopic 1,3,6,8-tetrakis(p-benzoate)pyrene linkers and has a scu network topology.

4. The method of claim 2, wherein the bismuth nodes Bi₆ nodes and the organic linkers comprise biphenyl groups.

5. The method of claim 1, wherein the patient is a human.

6. A contrast composition comprising: a metal-organic framework comprising bismuth nodes connected by multitopic organic linkers and at least one carrier to a patient, wherein the carrier comprises a sugar, a polysaccharide, a starch, or a mixture of two or more thereof.

7. The contrast composition of claim 6, wherein the bismuth nodes are Bi₆ nodes.

8. The composition of claim 7, wherein the carrier comprises lactose, dextrose, saccharose, cellulose, dextran, carboxydextran, aminated dextran, starch, chitosan, or a combination of two or more thereof.

9. A metal-organic framework comprising a permanently porous crystalline material comprising Bi₆ nodes connected by multitopic organic linker molecules.

10. The metal-organic framework of claim 9, wherein the multitopic organic linkers comprise pyrene groups.

11. The metal-organic framework of claim 10, wherein the multitopic organic linkers comprise tetratopic 1,3,6,8-tetrakis(p-benzoate)pyrene linkers and the metal-organic frameworks have an 8-connected scu network topology.

12. The metal-organic framework of claim 10, wherein the multitopic organic linkers comprise tetratopic 4,4′,4″,4′″-(pyrene-1,3,6,8-tetrayl)tetrabenzoic acid (TBAPy) linkers and the metal-organic frameworks have a csq network topology.

13. The metal-organic framework of claim 9, wherein the multitopic organic linkers comprise bipyridine groups.

14. The metal-organic framework of claim 13, wherein the multitopic organic linkers comprise tetratopic 3,3′,5,5′-tetrakis(4-carboxyphenyl)-1,1′-biphenyl linkers and the metal-organic frameworks have a csq network topology.
